(19) United States
(12) Patent Application Publication
(10) Pub. No.: US 2023/0062326 A1
(43) Pub. Date: Mar. 2, 2023

(54) GUIDED REHABILITATION TO RELEARN MOTOR CONTROL USING NEUROMUSCULAR ELECTRICAL STIMULATION

(71) Applicant: Battelle Memorial Institute, Columbus, OH (US)

(72) Inventors: Samuel COLACHIS, Columbus, OH (US); Lauren WENGERD, Columbus, OH (US)

(21) Appl. No.: 17/882,263

(22) Filed: Aug. 5, 2022

Related U.S. Application Data
(60) Provisional application No. 63/236,843, filed on Aug. 25, 2021.

(51) Int. Cl. A61N 1/36; A61N 1/04

(52) U.S. Cl. CPC A61N 1/36003; A61N 1/0492; A61N 1/0452; A61N 1/0456; A61N 1/36031; A61N 1/36034

(57) ABSTRACT

In rehabilitation, a stimulation pattern when applied to a body part by a neuromuscular electrical stimulation (NMES) device is effective to cause the body part to perform an intended action. The applying includes increasing a stimulation level at which the stimulation pattern is applied over time and, during the applying, acquiring video of the body part. The body part is monitored during the applying by analysis of the video, and the applying is automatically stopped in response to the monitoring indicating the body part has performed the intended action. The stimulation pattern may be defined as one or more subsets of electrodes of the NMES device and an electrode group stimulation level for each respective subset of electrodes, and the increasing of the stimulation level comprises increasing a scaling factor applied to the electrode group stimulation levels over time.

This application claims the benefit of U.S. Provisional Application No. 63/236,843 filed Aug. 25, 2021 and titled “GUIDED REHABILITATION TO RELEARN MOTOR CONTROL USING NEUROMUSCULAR ELECTRICAL STIMULATION”, which is incorporated herein by reference in its entirety.

### BACKGROUND

The following relates to the rehabilitation therapy arts, to physical therapy arts, to the relearning of muscular control in patients with traumatic brain injury (TBI), Alzheimer's disease, brain lesions, stroke, spinal cord injury, or another neurological disorder, and to the like.

Rehabilitation therapy is a crucial recovery component for numerous medical conditions. For example, every year, more than 200,000 Traumatic Brain Injury (TBI) cases are reported in the United States alone. Many patients with TBI suffer cognitive impairment that affects their ability to interact with their environments and objects of daily living, preventing them from living independently. Approaches for TBI rehabilitation includes mirror therapy and therapist guided exercises. Since TBI is such a diffuse injury, these therapies only help some patients, and require therapist time which may be limited by insurance reimbursement or other practical considerations. More generally, rehabilitation therapy is commonly employed in persons suffering from agnosia (difficulty in processing sensory information) or apraxia (motor disorders hindering motor planning to perform tasks). Besides TBI, these conditions can be caused by conditions such as Alzheimer's disease, brain lesions, stroke, or so forth.

Colachis et al., U.S. Pub. No. 2021/0082564 A1 titled “Activity Assistance System” is incorporated herein by reference in its entirety. This reference discloses an activity assistance system that includes a video camera arranged to acquire video of a person performing an activity. An output device outputs human-perceptible prompts, and an electronic processor is programmed to execute an activity script. The script comprises a sequence of steps choreographing the activity. The execution of each step includes presenting a prompt via the output device and detecting an event or sequence of events subsequent to the presenting of the prompt. Each event is detected by performing object detection on the video to detect one or more objects depicted in the video and applying one or more object-oriented image analysis functions to detect a spatial or temporal arrangement of one or more of the detected objects. Each event detection triggers an action comprising at least one of presenting a prompt via the output device and and/or going to another step of the activity script.

Certain improvements are disclosed herein.

### BRIEF SUMMARY

In accordance with some illustrative embodiments disclosed herein, a rehabilitation system is disclosed, which includes at least one sensor configured to monitor movement of a body part, a neuromuscular electrical stimulation (NMES) device configured to be worn on the body part and having electrodes arranged to apply NMES to the body part when the NMES device is worn on the body part, and an electronic processor. The electronic processor is programmed to: obtain a stimulation pattern that when applied to the body part by the NMES device is effective to cause the body part to perform an intended action; apply the stimulation pattern to the body part using the NMES device; and stop the application of the stimulation pattern to the body part in response to the at least one sensor indicating the body part has performed the intended action. In some embodiments, the electronic processor is programmed to apply the stimulation pattern to the body part using the NMES device with the ramping stimulation level by: applying the stimulation pattern to the body part using the NMES device at an initial stimulation level that is too low to produce functional electrical stimulation of the body part; and, in response to the at least one sensor indicating the body part is not performing the intended action with the stimulation pattern applied to the body part at the initial stimulation level, increasing the stimulation level above the initial stimulation level. In some embodiments, the at least one sensor comprises a video camera arranged to acquire video of a body part.

In accordance with some illustrative embodiments disclosed herein, a rehabilitation method comprises: obtaining a stimulation pattern that when applied to a body part by an NMES device is effective to cause the body part to perform an intended action; applying the stimulation pattern to the body part using the NMES device, wherein the applying includes increasing a stimulation level at which the stimulation pattern is applied to the body part with increasing time; during the applying, acquiring video of the body part; monitoring the body part during the applying by analysis of the video of the body part performed by an electronic processor; and automatically stopping the applying in response to the monitoring indicating the body part has performed the intended action. In some embodiments, the stimulation pattern comprises one or more subsets of electrodes of the NMES device and an electrode group stimulation level for each respective subset of electrodes, and the increasing of the stimulation level with increasing time comprises increasing a scaling factor applied to the electrode group stimulation levels over time.

In accordance with some illustrative embodiments disclosed herein, a non-transitory storage medium stores instructions readable an executable by an electronic processor to perform a rehabilitation method including: applying a stimulation pattern to a body part using an NMES device; during the applying, analyzing video of the body part to determine whether the body part has performed an intended action; and automatically stopping the applying in response to the analysis of the video indicating the body part has performed the intended action. In some embodiments, the applying includes: applying the stimulation pattern at an initial stimulation level that is insufficient to produce functional electrical stimulation of the body part; and in response to the analysis of the video during the applying of the stimulation pattern at the initial stimulation level indicating the body part has not performed the intended action, applying the stimulation pattern at a stimulation level that is higher than the initial stimulation level and that is sufficient to produce functional electrical stimulation of the body part. The latter applying of the stimulation pattern at a stimulation level that is higher than the initial stimulation level and that is sufficient to produce functional electrical stimulation of the body part may include ramping up the stimulation level to increase the functional electrical stimulation of the body part over time.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically shows a motor control rehabilitation system.

FIG. 2 diagrammatically shows a motor control rehabilitation method suitably performed using the system of FIG. 1.

FIG. 3 diagrammatically shows a motor control rehabilitation method suitably performed using the system of FIG. 1 in further detail.

FIG. 4 presents an experimental test and results of that experimental test as described herein.

### DETAILED DESCRIPTION

The activity assistance system of Colachis et al., U.S. Pub. No. 2021/0082564 A1 previously referenced advantageously provides a person suffering from TBI or another neurological disorder with the ability to autonomously practice activities of daily living, without (or with minimal) assistance of a (human) physical therapist. This has a number of advantages, such as reducing the workload of specially trained physical therapists, and enabling the person to perform rehabilitation as often as desired, in a private setting, and in some cases in the person's own residence.

However, it would be advantageous to provide more detailed assistance in relearning motor control for performing specific actions, such as grasping an object. In a rehabilitation setting, and with the assistance of a physical therapist, a TBI or stroke victim can sometimes relearn motor control for performing volitional actions such as grasping an object on his or her own. Relearning volitional motor control for performing activities of daily living can enable the TBI or stroke victim to recover the ability to engage in independent living, or at least to require less assistance on a daily basis. The physical therapist providing guidance in such relearning usually has extensive training in rehabilitation therapy. The volitional motor control relearning process usually requires many one-on-one physical therapy sessions performed over an extended period of time, which occupies valuable time of the physical therapist.

Furthermore, the physical therapist can provide only approximate guidance to the person in relearning volitional motor control for performing various manual tasks. Even a relatively simple task such as grasping an object entails coordinated motor control of many different muscles. A human hand has 14 digit joints (four fingers with three joints each, and a thumb with two joints) and additional muscles in the palm involved in grasping, as well as muscles in the wrist for rotating and otherwise orienting the hand. A grasping action requires coordination of many or all of these muscles. A physical therapist can provide general guidance, such as verbally guiding the person in performing an action, and/or manually positioning the fingers of the person's hand in a target (e.g. grasp) position to help the person relearn how to form that hand position. However, the physical therapist usually cannot provide guidance to the person in real-time specifically targeting specific muscles that need to contract to perform a given volitional motor control action.

Embodiments disclosed herein provide overcome these deficiencies and provide muscle-specific guidance for relearning volitional motor control. The guidance is provided automatically, and is designed to dynamically adjust over time as the person relearns volitional motor control. The automated guidance also provides assistance, to the extent needed, to ensure success in performing an action, thus encouraging the person to continue with the relearning process.

More particularly, rehabilitation systems and techniques disclosed herein leverage neuromuscular electrical stimulation (NMES) and functional electrical stimulation (FES) to provide individually tailored assistance at the level of individual muscle units for assisting a person suffering from a neurological disorder in relearning volitional motor control for performing specific actions, such as those involved in performing activities of daily living.

The disclosed motor control rehabilitation approaches repurpose a device for providing FES, which typically has the form factor of a sleeve or other garment that is worn by a person, and includes surface electrodes contacting the skin of the wearer for delivering FES. A stimulation amplifier is built into or connected with the FES device to apply electrical stimulation to muscles of the arm, leg, or other anatomy on which the FES device is disposed to stimulate muscle contraction and consequent motion of an arm, leg, hand, or other body part. Bouton et al., U.S. Pub. No. 2018/0154133 A1 titled “Neural Sleeve for Neuromuscular Stimulation, Sensing and Recording”, and Bouton et al., U.S. Pub. No. 2021/0038887 A1 titled “Systems and Methods for Neural Bridging of the Nervous System”, both of which are incorporated herein by reference in its entirety, provide illustrative examples of such FES devices. These references also disclose illustrative applications for assisting patients with spinal cord injury, stroke, nerve damage, or the like. In some approaches there disclosed, a cortical implant receives neural signals from the brain which are decoded to detect an intended action which is then carried out by FES of the muscles of the anatomy (e.g. arm and/or hand). Sharma et al., U.S. Pub. No. 2020/0406035 A1 titled “Control of Functional Electrical Stimulation using Motor Unit Action Potentials”, which is incorporated herein by reference in its entirety, discloses an approach in which surface electromyography (EMG) signals are measured using the FES device. Motor unit (MU) action potentials are extracted from the surface EMG signals and an intended movement is identified from the MU action potentials. FES is delivered which is effective to implement the intended movement. This reference also discloses an illustrative FES device in the form of a sleeve designed to be worn around the forearm of a person undergoing volitional motor control relearning, with around 50-160 or more electrodes in some embodiments to provide high-density electromyography (HD-EMG).

In embodiments disclosed herein, such an FES device is repurposed as a device for assisting a person with a neurological disorder in relearning volitional motor control, that is, to relearn how to volitionally use his or her own muscles to perform body movements implementing actions of activities of daily living or the like, such as grasping an object using the person's own hand. To do so, initial NMES at a low stimulation level (and in some cases at a level insufficient to cause muscle contractions producing movement) is initially applied to prompt the person to perform the movement. The initial NMES is applied with the same pattern that, at a higher stimulation level, would produce the desired movement by FES. Although the initial NMES may be insufficient to produce muscle contractions, the initial NMES is sensible by the person as a tingling or other sensation of the muscles (or, more precisely, of sensory nerves associated with those muscles) that the person needs to volitionally control to perform the action. Machine vision techniques (e.g., performed via video analysis) are used to automatically assess how well the hand action or other action implemented by a body movement is proceeding (or indeed if it is proceeding at all) during the initial NMES. Depending on the level of neurological deficiency and the level of the initial NMES, the action may not be occurring at all, or body movement may occur but be unsatisfactory for performing the action. For example, in an intended grasping movement, one or more fingers may be opening rather than closing on the object, and/or the finger movement detected by the video analysis may be in the correct direction toward closing on the object but insufficient to produce grasping, for example stopping before the fingers actually contact and apply force to the object.

If movement is undetectable in the video analysis or is unsatisfactory, the system then increases the NMES stimulation level so as to stimulate muscle contraction. At this point the NMES becomes FES, i.e. functional electrical stimulation that is functioning to stimulate muscle contraction. (As used herein, NMES encompasses any type of neuromuscular stimulation, while FES more narrowly encompasses NMES at a stimulation level sufficient to stimulate observable muscle contraction). In some embodiments, the FES stimulation level is slowly increased to encourage the person to implement the movement by volitional control of his or her own muscles. Thus, the desired movement may be obtained by combination of the FES and the person's own volitional control. If the person is unable to produce volitional control of his or her muscles even under the ramped FES, eventually the FES stimulation level increases to a level sufficient to cause the musculature to implement the intended action under only FES control.

The disclosed approaches for assisting a person in relearning volitional muscle control for performing an action have numerous advantages. For example, the initial NMES provides a perceptible stimulation at the muscles that need to contract in order to perform the intended action. This prompts the person's specific muscles needed to perform the action—something that cannot be provided even by a skilled human physical therapist. Furthermore, by slowly ramping up the stimulation level of the subsequent FES, the person is encouraged to attempt the movement on his or her own, again under the guidance of the FES. In the initial stages of motor control relearning, the person may be able to provide only limited efferent neural signal to the muscles, or none at all; or the provided efferent neural signal may be incorrect (e.g., stimulating an incorrect muscle action). As the relearning progresses, the person's ability to send correct efferent neural signal to the correct muscles increases, and an increasing portion of the movement for performing the action is generated by motor control of the person rather than by the FES. Still further, as the process is mostly or fully automated, the person can practice anytime he or she wishes. This enables the person to gradually rebuild the ability of the brain motor cortex to send the correct efferent neurological signals to the correct muscle units in order to perform the intended action, thereby relearning volitional motor control. A further advantage is that the level of assistance is directly quantifiable as the stimulation level of the maximum applied FES during the movement, thus providing quantitative scoring information by which a doctor or other medical professional can evaluate the person's progress in relearning volitional motor control. Yet a further advantage is that since the person always ultimately achieves the desired action (even if wholly by way of the FES), the person experiences a sense of achievement, and is thereby encouraged to continue the volitional motor control relearning process.

In some embodiments, a controlled high-definition NMES sleeve (or, more generally, an NMES garment) worn on the forearm (or other body part for which volitional motor control is to be learned) evokes hand actions (or more generally, actions) to complete each step of a functional activity based on real-time activity performance. For example, objects the person intends to grab, as well as the person's hand or hands, are detected via video-based object detection or object tracking alternatives (ex. RFID), and based on human-object interaction information identified by the rehabilitation system, associated grips are evoked and terminated via the NMES garment. The rehabilitation system actively guides a stroke survivor or other person with a neurological disorder through clinically relevant, functional activities using human-object interaction detection to control high-definition, moment-to-moment electrical stimulation of the forearm to evoke movements necessary to manipulate objects. As the person undergoing volitional motor control relearning practices the activity and their activity performance improves (as autonomously quantified by the video and optionally other sensor feedback), the level of FES assistance decreases, enabling the person to become more independent over time through a rehabilitation method called neuromuscular reeducation.

The disclosed approaches empower the patient by creating a closed-loop interactive rehabilitation training platform where performance-based interactions with objects are provided physically to the body instead of through words and actions from a physical therapist. Additionally, the system can be used to measure activity performance for progress monitoring and patient insight. The disclosed approaches use human-object interaction detection during multi-step activities as a control mechanism for FES and upper limb reanimation, and FES assistance modulation based on activity performance, quantified by the video (and optionally other sensor) feedback.

Some illustrative examples include an object detection system, comprising a camera to capture live-stream video of the activity scene. Object detection operates by use of a convolutional neural network (CNN) framework customized with transfer learning to identify relevant objects (such as a coffee cup, toothbrush, eating utensil, et cetera). Grabbable objects as well as the person's hands are detected in the activity scene. The person undergoing relearning is prompted via a human perceivable prompt to perform a step of an activity. Machine vision or the like is used to detect when the person's hand is near a given object and/or if a step of the activity has been completed. The object the hand is closest is, in one approach, used to determine what type of grip is required to grip the object. Or if the person is prompted to transfer an object, once the object has been transferred, the cued grip would be released. Information about the sequence is fed to a NMES garment worn on the person's forearm to cue the necessary electrode activation pattern for evoking the necessary grip. A database is, in one approach, used to pair objects to grips. In some embodiments, an activity script is followed, in which the rehabilitation system detects when a prompted event has been completed, performs the necessary actions, and prompts a subsequent step. In one case, the actions may be to initiate and terminate FES to evoke specific movement. As the person undergoing volitional motor control relearning performs steps of the activity, performance metrics are recorded and used for performance summary reports and real-time interactions. As the person modulates their activity performance (gets better or worse), the amount of FES assistance modulates accordingly. For example, if the person becomes better at a task, the FES amplitude will decrease, providing more autonomy to the person. This cycle continues until the activity or sequence of activities are complete.

In illustrative embodiments of the disclosed volitional motor control rehabilitation, an accurate real-time assessment of the progress of the body movement is determined using computer vision, optionally augmented by other types of sensors. Advantageously, the computer vision can also optionally provide other functionality such as determining the type of action the person intends to perform. For example, in the case of a hand movement, computer vision can assess the position of hand relative to the object to be grasped, and use this information to trigger the NMES.

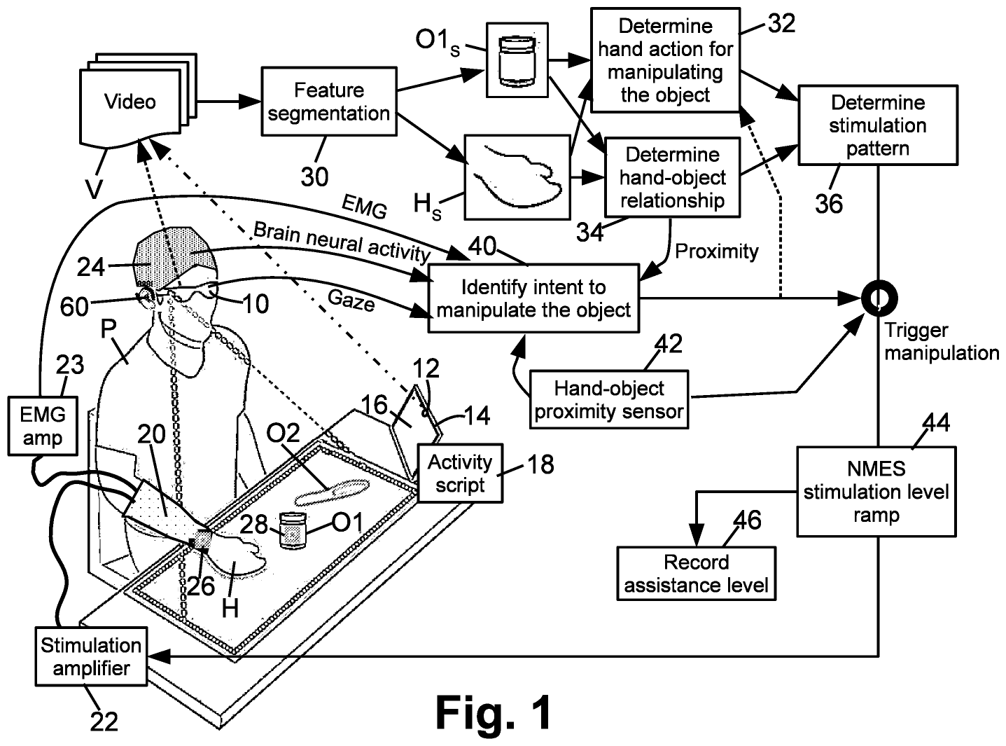

With reference to FIG. 1, a non-limiting illustrative example of a system is shown for providing volitional motor control rehabilitation to a person P suffering from a traumatic brain injury (TBI), Alzheimer's disease, brain lesions, stroke, spinal cord injury, or another neurological disorder. The person P wears smart glasses 10 having an eyeglasses form factor and that include a video camera for acquiring video V of an object (for example, a jar O1 or a knife O2) and a hand H. For example, the smart glasses 10 may be Google Glass™. Instead of using a camera of the smart glasses 10 to acquire the video V, a camera 12 of a computer 14 having a display 16 may be used to acquire the video V. Optionally, the computer 14 or another user dialoging device provides an activity script 18 of actions the person P is to perform in the rehabilitation therapy. For example, in a rehabilitation system for relearning volitional motor control to perform an activity of daily life (ADL), the computer 14 may provide instructions by way of the activity script 18 to the person P for performing a sequence of actions making up the ADL. As an example, the sequence of actions of the activity script 18 may include grasping the knife O2, opening the jar O1, and using the knife to obtain a portion of peanut butter (or other content) of the jar.

The patient P also has a neuromuscular stimulation (NMES) sleeve 20 configured to be worn on the hand and/or on an arm to which the hand H is attached. The illustrative NMES sleeve 20 is configured to be worn on the hand H and/or an arm of the person P. The NMES sleeve 20 has surface electrodes (not shown) arranged on an inner surface of the sleeve 20 to electrically contact the hand and/or arm when the sleeve is worn on the hand and/or the arm of the person P. It will be appreciated that if a different action performed by another body part is to be relearned then a NMES device of a different garment shape may be used, e.g. an NMES device with a legging form factor worn on a leg to relearn actions performed by the leg.

A stimulation amplifier 22 is connected to apply NMES to muscles of the hand or arm via the surface electrodes of the NMES sleeve 20. If the NMES stimulation level is high enough then functional electrical stimulation (FES) is performed to stimulate muscle contractions and consequent movement of the fingers, thumb, or other hand movements. On the other hand, if the applied NMES is at a lower stimulation level then it may not stimulate muscle contractions, but only produce a tingling or other perceptible sensation in the stimulated muscles.

Various training approaches can be used to map surface electrodes to muscle groups or muscle units of the hand and/or arm in order to enable controlled stimulation of specific muscle groups or units to evoke sensation (in NMES at low stimulation level) or specific movements (in FES; that is, in NMES at stimulation level that is sufficiently high to produce observable muscle contractions). The NMES sleeve 20 may, for example, be designed to be worn around the forearm of the person P (possibly including the wrist, and possibly further extending to encompass a portion of the hand H), and may in some embodiments have around 50-160 or more electrodes to provide high-density stimulation, and optionally also high-density electromyography (HD-EMG) using a suitable EMG amplifier 23.

The patient P may have other optional monitoring devices, such as an illustrative optional skullcap 24 with surface electrodes (not shown) on its inner surface that contact the scalp of the patient P when worn. The surface electrodes of the skullcap 24 may serve as EEG electrodes for acquiring EEG signals, or may perform brain neural activity measurement that is input to a BCI (not shown). The smart glasses 10 may optionally include gaze trackers that, in conjunction with the video V acquired by the camera of the smart glasses 10, enables identification of an object that the eyes of the person P are focused on. For example, if the person looks intently at object O1 then the gaze tracking will measure the direction of the eyeballs and thus detect the point in space the gaze is focused at, and by mapping that to the video V the gaze can be identified as looking at the object O1.

Still further, the rehabilitation system may include tracking tags, such as an illustrative radiofrequency identification (RFID) tag 26 attached to the NMES sleeve 20 at its closest point to the hand H (thereby usable to track the location of the hand H), and an RFID tag 28 attached to the object O1. With this approach and with two, and more preferably at least three, RFID reader stations enabling triangulation of the signal from the RFID tags in space, the RFID tags 26, 28 can enable detection of the proximity of the hand H to the object O1 at any time.

An electronic processor is programmed by instructions stored on a non-transitory storage medium (components not shown) to perform the various data processing as described herein, such as: feature segmentation 30 to extract a segmented hand Hs corresponding to the hand H and a segmented object O1s corresponding to the object O1 closest to the hand H; determination 32 based on the segmented object O1s (and optionally also the segmented hand Hs) of a hand action for manipulating the object (for example, using a lookup table of hand gripping actions for different types of objects); determination 34 based on the segmented hand Hs and segmented object O1s of a hand-object spatial relationship (e.g. proximity of the hand H to the object O1, or a more detailed hand-object relationship indicating orientation of the hand H respective to the orientation of the object O1, or an even more complex hand-object spatial relationship such as indicating by vectors in three-dimensional space the location of the hand and object, et cetera); and determination 36 of an NMES stimulation pattern for implementing the determined hand action for manipulating the object by FES. Note that whether the NMES with the determined NMES stimulation pattern actually produces the hand action depends on the stimulation level of the applied NMES. The stimulation level of the applied NMES can be varied, and if it is low enough then the applied NMES may produce tingling or other sensation in the stimulated muscles but may not produce actual muscle contractions effective for performing the hand action. In general, the stimulation pattern is suitably defined as a set of voltages (or electrical currents or other electrical parameters) applied to selected electrodes of the NMES device 20 to implement the determined hand action.

The electronic processor is optionally further programmed by the instructions stored on the non-transitory storage medium to perform an operation 40 in which an intent to manipulate the object is determined. Various approaches can be used. In one approach, the gaze as determined by gaze trackers of the smart glasses 10 is used to identify the person P is staring at the object O1 for a predetermined time interval (e.g., 5 seconds, as a non-limiting example) and based on that steady gaze it is inferred that the person P wants to grasp and/or move the object O1. As another example, brain neural activity measured by the skullcap 24 is decoded by a BCI to determine the intent to manipulate the object. In another embodiment, proximity of the hand H to the object O1 is measured by a hand-object proximity sensor 42 (for example, RFID tag readers that read the RFID tags 26, 28 to determine the locations of the hand H and object O1 and the distance therebetween), or is determined from the hand-object relationship determined at processing operation 34. Advantageously, the determination of the intent to manipulate the object can be at a generalized level, and the operation 40 is not required to determine the detailed hand grip action that is intended—rather, that is determined by the computer vision processing 30, 32, 34 performed on the video V. Thus, for example, BCI determination of this general intent is more reliable than attempting detailed determination of the specific hand grip action that is intended.

It is noted that in some embodiments for relearning volitional motor control, there may be no need to determine the intent to manipulate the object. For example, if the computer 14 provides the optional activity script 18 that the person P is being instructed to follow, then the intended object manipulation may be assumed to be the object manipulation called for in the step of the activity script 18 currently being performed.

The operation 40 may also (or alternatively) operate in real-time to identify a trigger, that is, the moment (or time interval) in time that the person P intends to perform the hand grip action or other object manipulation action. For example, this trigger can be based on proximity of the hand H to the object O1 measured in real-time using the proximity sensor 42 or the hand-object relationship determined in real-time by iterative repetition of the operation 34 on successive frames of the video V. When the hand closes to within a predetermined distance of the object (which may be as small as zero in some specific examples) then the action is triggered, and the stimulation pattern determined in the operation 36 is applied by the stimulation amplifier 22 to cause the NMES device 20 to begin to apply NMES to the muscles of the hand H.

To implement the gradual increase of NMES to provide automated graduated relearning of motor control as disclosed herein, the electronic processor is further programmed to implement an NMES stimulation level ramp 44 in which the stimulation pattern determined at operation 36 is applied at a controlled stimulation level. For example, the initial NMES may be at a stimulation level insufficient to stimulate muscle contraction, but is sensed by the person P as a tingling or other sensation in the muscles of the hand or wrist that are to be contracted to perform the hand action. If the motor control has been sufficiently learned, this may cue the person P to perform the volitional motor control to implement the hand action without any FES assistance. On the other hand, if the motor control has been learned to a lesser degree, then the person's attempt at volitional motor control may produce some muscle contraction, but not enough to implement the hand action. In another instance, the person's attempt at volitional motor control may produce some muscle contraction, but with some incorrect muscle contractions, such as causing a finger to uncurl rather than curling onto the object to grip it. In these cases, the NMES ramp 44 continues to ramp the NMES stimulation level until some FES is produced that assists the person's volitional motor control in completing the hand action. As yet another possible case, if the motor control has not yet been learned at all, then the NMES ramp 44 continues to ramp the NMES stimulation level until sufficient FES is produced to perform the hand action entirely by FES, without any contribution from the person's volitional motor control.

The NMES stimulation level ramp 44 may suitably operate as follows. As a non-limiting illustrative example, assume the stimulation pattern determined at the operation 36 includes: applying a voltage V1 to an electrode group E1; applying a voltage V2 to an electrode group E2; and applying a voltage V3 to an electrode group E3. The electrode groups E1, E2, and E3 are suitably groups of the electrodes of the high density array of electrodes of the NMES sleeve 20. The various electrode groups E1, E2, E3 may be mapped to corresponding muscles or muscle units. As an example, the electrode group E1 may stimulate an index finger movement, the electrode group E2 may stimulate a middle finger movement, and the electrode group E3 may stimulate a thumb movement, and the combined movements of the index finger, middle finger, and thumb are effective to grasp an object. The voltages V1, V2, and V3 represent stimulation levels as voltages, and may in general be different voltage levels. Depending on the design of the stimulation amplifier 22 and circuitry of the NMES sleeve 20, the electrical stimulation level may be specified by another electrical parameter such as an electrical current, a pulse width in the case of pulse width modulation (PWM) control of the stimulation level, or some combination of such electrical parameters.

To implement the NMES stimulation level ramp 44, in one approach a stimulation level scaling factor F is applied, so that the electrode group E1 is energized by a voltage F*V1, the electrode group E2 is energized by a voltage F*V2, and the electrode group E2 is energized by a voltage F*V3. It will be appreciated that if F=0 then no NMES is applied (or, equivalently, the stimulation level is zero). Furthermore, if the voltages V1, V2, and V3 of the stimulation pattern determined at operation 36 are sufficient to stimulate the respective hand digits to perform the intended hand action with no assistance from the volitional motor control of the person P, then if F=1 this would provide FES execution of the intended hand action with no assistance from the person P. In some cases, the ramp might beneficially ramp up to a value slightly higher than F=1. For example, if the person's volitional motor control is incorrectly causing a finger to uncurl when it should be curling to perform the intended hand action, then a value of F>1 might be needed to offset that uncurling force. However, ramping significantly above F=1 is generally avoided as values of F significantly higher than 1 can generate pain, and/or damage the stimulated muscle.

It will be appreciated that the foregoing can be generalized to the following process. The electronic processor is programmed to determine the stimulation pattern (operation 36) as one or more subsets of electrodes of the NMES device (e.g., subsets E1, E2, and E3 in the example) and an electrode group stimulation level for each respective subset of electrodes (e.g., voltages V1, V2, and V3 in the example). The electronic processor is further programmed to apply the stimulation pattern to the body part using the NMES device 20 with the ramping stimulation level 44 implemented as a ramping of a scaling factor applied to the electrode group stimulation levels (e.g., the scaling factor F in the example). It is noted that a subset of the electrode may be as small as a single electrode in some embodiments.

Thus, the NMES stimulation level ramp increases steadily until either the hand action is accomplished, or until F reaches some maximum value Fₘₐₓ that is chosen to ensure the FES does not produce pain and/or muscle damage. (In some cases, Fₘₐₓ=1 may be the chosen maximum value). In some embodiments, the electronic processor is further programmed to perform an operation 46 in which the maximum NMES applied in the ramp 44 is recorded. This maximum NMES stimulation level can for example be recorded as the maximum applied value of the stimulation level scaling factor F, and advantageously provides a quantitative metric of the extent to which the person P has relearned the volitional motor control being performed. For example, if the maximum NMES as quantified by F is below the threshold for producing functional electrical stimulation producing muscle contraction, then this indicates the person P has fully relearned the volitional muscle control. On the other hand, if F is near 1 or has reached the maximum value Fₘₐₓ, then this indicates the person P has not relearned the volitional muscle control to any significant extent.

Optionally, the recordation 46 may be more elaborate. For example, the video sequence of the hand action over the course of the NMES stimulation level ramp may be recorded as an MPEG video clip or the like, with the frames of the video annotated with the value of F at each frame. This provides more information to a medical professional reviewing the rehabilitation session, as the medical professional may observe (as a nonlimiting example) that the person initially had some volitional motor control that partially closed the hand, but that was insufficient to complete the hand action, after which the ramping FES reached a level sufficient to assist the person in completing the action.

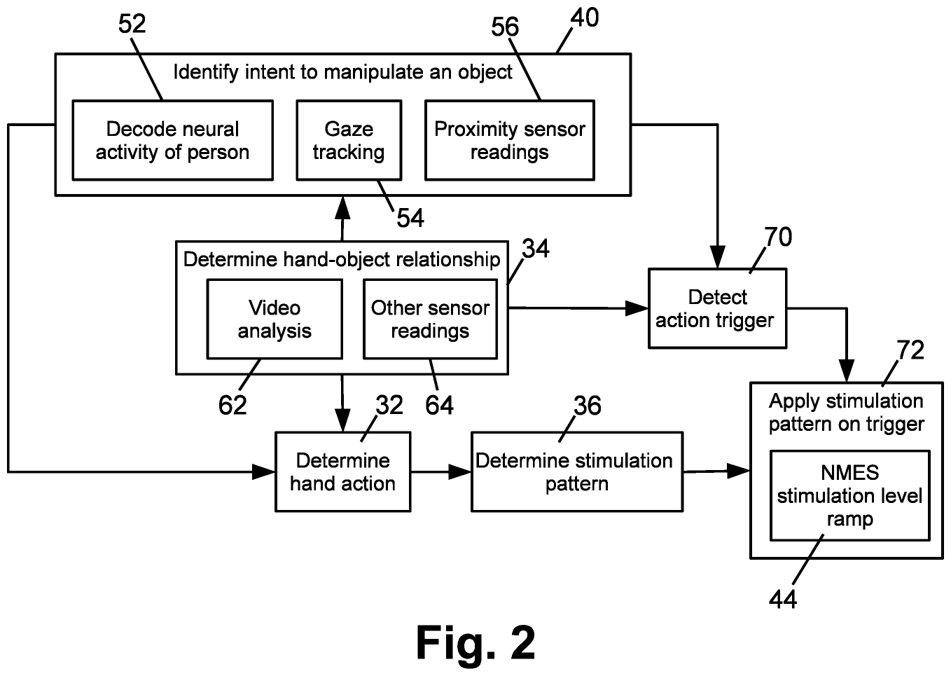

With reference now to FIG. 2, a method suitably performed using the system of FIG. 1 is described, in which the intended hand action is automatically determined. In the operation 40 also shown in FIG. 1, an intent to manipulate an object is identified. This may be done by various ways. In one illustrative approach 52, neural activity of the person measured by surface electrodes of the skullcap 24 (or, in another embodiment, measured using implanted electrodes) is decoded to identify the intent. For example, the operation 52 can employ a support vector machine (SVM) trained to receive brain neural activity and decode an intended action. See Bouton et al., U.S. Pub. No. 2021/0038887 A1 titled “Systems and Methods for Neural Bridging of the Nervous System” which is incorporated herein by reference in its entirety. Other types of machine learning (ML) can be employed for the decoding, such as deep neural network (DNN) decoders. As previously note, when using the system of FIG. 1 which employs computer vision to determine the specific hand action for implementing the intended action, the intend decoding performed in the operation 52 advantageously need only identify the general intent of the person, rather than a detailed intent with respect to specific muscles of the hand.

Another illustrative approach for identifying the intent 40 employs gaze tracking 54 using eye trackers of the smart glasses 10 to identify the intent. For example, the eye trackers identify the person is focusing at a point in space, and maps this focus point to a location in the video V (in this case, preferably acquired by a video camera of the smart glasses 10 so that the video V is spatially registered with the gaze tracking). If the person focuses on a given object (e.g. the object O1) for a predetermined time interval (e.g., 5 seconds as a nonlimiting example) then an intent to manipulate that object is identified. Again, due to the use of computer vision to determine the detailed hand interaction, it is sufficient to identify the general intent to manipulate the object, which is feasibly achieved using gaze tracking 54.

Yet another illustrative example for identifying the intent 40 employs proximity sensor readings 56, such as those from the RFID tags 26, 28, to identify intent to manipulate an object. For example, consider a case in which the person P has volitional control of the upper arm muscles so that the person P can move the hand H toward the object O1. This could be the case, for example, if the person P has a prosthetic hand attached to an otherwise functional arm, or if the person has suffered a stroke or spinal cord injury which has left the hand H partially or entirely paralyzed, but in which the person P retains volitional control of the upper arm muscles. In such a case, the proximity sensors 26, 28 suitably detect when the person P moves the hand H toward the object O1 (for example), and infers intent to manipulate the object from that movement. The inference of intent can be based on a distance between the object O1 and the hand H becoming less than a predetermined threshold. Additionally or alternatively, the inference of intent can be based on the velocity of the hand H, e.g. a rapid movement of the hand H toward the object O1 can provide information from which the intent is inferred.

It is to be appreciated that the foregoing illustrative approaches can optionally be combined to infer the intent to manipulate the object. For example, a weighted combination of intent from neural activity decoding and gaze tracking can be combined, and the intent is identified only if both of these indicate the same intent to manipulate the same object. Moreover, additional or other information indicative of intent to manipulate an object can be used in the intent identification 40, such as EMG signals acquired using the electrodes of the NMES sleeve 20 if the sleeve has EMG measurement capability (e.g., as exemplified by illustrative EMG amplifier 23).

With continuing reference to FIG. 2, at an operation 34 (also shown in FIG. 1) a hand-object relationship is determined. In some embodiments, the operation 34 is triggered by the operation 40, that is, once an intent to manipulate a specific object has been identified, then the operation 34 is performed to identify the hand-object relationship. Alternatively, for some tasks the operation 34 can be performed independently of the operation 40. For example, if the system of FIG. 1 is providing assistance for an activity of daily living in accordance with the activity script 18, in which there is only a small, closed set of objects to be manipulated (e.g., in the case of making a peanut butter-and-jelly sandwich, this closed set may include bread, a jar of peanut butter, a jar of jelly, a knife for the peanut butter, a knife for the jelly, and a plate) then the operation 34 may be performed to track the hand-object relationship for each of these objects. It is also noted that both operations 40, 34 may be performed continuously (that is, iteratively repeated) in order to identify intent to manipulate an object in real time (so that, for example, if the person P moves the hand H toward the jar O1 and then moves it toward the knife O2 the change in intent is detected in near real-time) and in order to continuously monitor the hand-object relationship for each object of interest.

As shown in FIG. 2, the operation 34 of determining the hand-object relationship relies partially or entirely on video analysis 62. In one approach, object detection is performed on the video V, in the hand H and the object O1 of interest are delineated in a frame of the video V by a bounding box (BB). The location of the hand H or object O1 can then be designated as the center of the BB, and this may move as a function of time. For example, a convolutional neural network (CNN) may be trained to detect the hand H, and another CNN may be trained to detect each object O1, O2 of interest. In another approach, the operation 62 may identify the hand H and object O1 using instance segmentation, in which objects are delineated by pixel boundaries. Instance segmentation provides object orientation and high-detail resolution by detecting exact pixel-boundaries of the hand H and each object O1, O2 in frames of the video V. Various instance segmentation techniques can be employed, such as pixel classification followed by blob connectivity analysis, or instance segmentation using mask regional CNNs trained for specific object types (see He et al., “Mask R-CNN”, arXiv:1703.06870v3 [cs.CV] 24 Jan. 2018). Other object identification techniques such as blob detection and template matching can be used to identify the hand H and each object O1, O2.

With the hand H and object O1 identified in frames of the video V, their spatial relationship can be estimated. In some embodiments, the spatial relationship includes distance between the hand H and object O1, and optionally also their locations in three-dimensional (3D) space. If the video V is 3D video, for example acquired using a range-finding camera or stereoscopic camera, then the spatial relationship can be estimated with high accuracy both in terms of distance between the hand and object and their locations in 3D space. If the video V is a 2D video then these values can only be estimated with reduced accuracy, e.g. based on distances in the 2D image but without information on the third dimension (depth). This can still be useful if the depth can be estimated in other ways—notably, most objects are manipulated with the arms extended with the elbows bent slightly, so that manipulated objects are at “arm's length”. This distance is about the same for persons of widely ranging size, and can optionally be measured for the specific person P using the system of FIG. 1 if greater accuracy is desired. Additionally or alternatively, the spatial relationship may include orientational information, such as the orientation of the hand H and the orientation of the object O1. This can be done with either 2D or 3D video, for example by fitting the image of the object to an a priori known shape model for the object to determine its orientation in space. With the orientation information it can be determined, for example, whether the hand H needs to be turned to have its palm facing toward the object O1 to pick it up.

In some embodiments, the hand-object relationship is determined in the operation 34 entirely by video analysis 62, that is, by applying computer vision techniques to frames of the video V to extract the spatial relationship between the hand H and object O1 for example. In other embodiments, the computer vision analysis 62 is augmented by other sensor readings 64, such as hand and/or object orientation information provided by at least one inertial measurement unit (IMU) secured to the hand and/or object, such as an accelerometer, gyroscope, magnetometer, or combination thereof. In some embodiments, an IMU may be embedded into or attached on the NMES sleeve 20 to provide information on hand orientation. It is also contemplated for the other sensor readings 64 to include information from bend sensors secured to fingers of the hand H or so forth.

The operations 40, 34 may be performed repeatedly, i.e. iteratively, to provide continuous updating of the intent and hand-object relationship. This information may be used by the system of FIG. 1 for various purposes. In an operation 32 (also shown in FIG. 1), a hand action is determined for performing the intended manipulation of the object identified in the operation 40. The operation 32 determines the appropriate hand action based on the hand-object relationship determined in the operation 34. Some common manipulations of an object include grasping the object, lifting the object, or moving the object. For any of these manipulations, the hand action includes an object grasping action for grasping the object. In one approach, the object grasping action is determined based on a shape of the object (e.g. jar O1) that is to be manipulated. This shape can be determined from the segmented object (e.g., O1s shown in FIG. 1). If the computer vision delineates a bounding box (BB) for the object, but not a detailed segmentation of the object, then a look-up table can be used to associate the object (for example, recognized using an image matching algorithm applied to the content of the BB) to an a priori known shape of the object. While grasping the object is a common manipulation, for which an object grasping action is an appropriate object interaction action, it is contemplated for the intended manipulation to be some other type of manipulation, such as pushing the object, and a corresponding object interaction action can be similarly determined for pushing the object or otherwise manipulating the object.

In addition to an object grasping action or other object interaction action, the overall hand action may further include a hand orientation action. For example, to grasp an object the palm of the hand must be facing the object prior to performing the object grasping action. Based on the relative orientation of the hand H and object O1 determined in the operation 34, an appropriate hand orientation action is also optionally determined. For example, the hand orientation action may suitably include rotating the hand at the wrist to rotate the palm into position facing the object. The hand action may also include other operations such as tilting the hand H up or down to align it with the object.

In an operation 36 (also shown in FIG. 1), a stimulation pattern is determined for implementing the hand action determined at the operation 32. This is suitably based on a pre-calibration of the NMES device 20, in which the stimulation pattern for producing specific hand movements is determined empirically and/or based on electrode-to-muscle mapping of the electrodes of the NMES device 20 to the underlying musculature anatomy. In a typical empirical approach, applied stimulation patterns are varied until the resulting measured or recorded hand configuration matches a target hand configuration, and this is repeated for each type of hand action to be pre-calibrated. The stimulation pattern determined at the operation 36 suitably includes both the electrode groups (e.g., E1, E2, and E3 of the previous example) and their respective stimulation values for producing the desired action (e.g., voltages V1, V2, and V3 of the previous example). However, when the stimulation pattern is applied it may be scaled by the stimulation level scaling factor F as previously described. The stimulation patterns for various hand actions determined by the pre-calibration are suitably stored in a non-transitory storage medium (which may be the same as, or different from, the non-transitory storage medium storing the instructions executed by the electronic processor) and can be retrieved in the operation 36 using a look-up table or the like associating hand actions with corresponding stored stimulation patterns.

In an operation 70, an action trigger is detected, and upon detection of the action trigger in an operation 72 the stimulation pattern determined at the operation 36 is applied. Various action trigger events or combinations of action trigger events can be used. In one example, the hand-object relationship determined at operation 34 is analyzed to determine when the distance of the hand H to the object O1 is within a predetermined threshold distance. This threshold might in some embodiments be zero, e.g. an object grasping action may be triggered when the video V indicates the hand has contacted the object (distance=0). Additionally or alternatively, EMG of the hand muscles measured using the NMES sleeve 20 and EMG amplifier 23 can be used to detect when the person P attempts to initiate muscle contractions for implementing the hand action. As previously described, the operation 72 may include the NMES stimulation level ramp 44 to start the application of the stimulation pattern at a low stimulation level (e.g., F close to zero in the previous nonlimiting illustrative example) that does not produce muscle contraction, and then ramp up the stimulation level until analysis of the video V shows the hand action is proceeding.

The examples of FIGS. 1 and 2 are directed to relearning motor control of the hand H. However, more generally the disclosed approaches can be used for relearning motor control for performing any action using any body part or anatomy whose volitional motor control has been compromised by a neurological disorder. For example, the body part could be a leg and the action could be a leg lift, a leg movement used during walking, or so forth. In this case the NMES sleeve 20 is sized and fitted to go on the patient's leg and/or lower torso region.

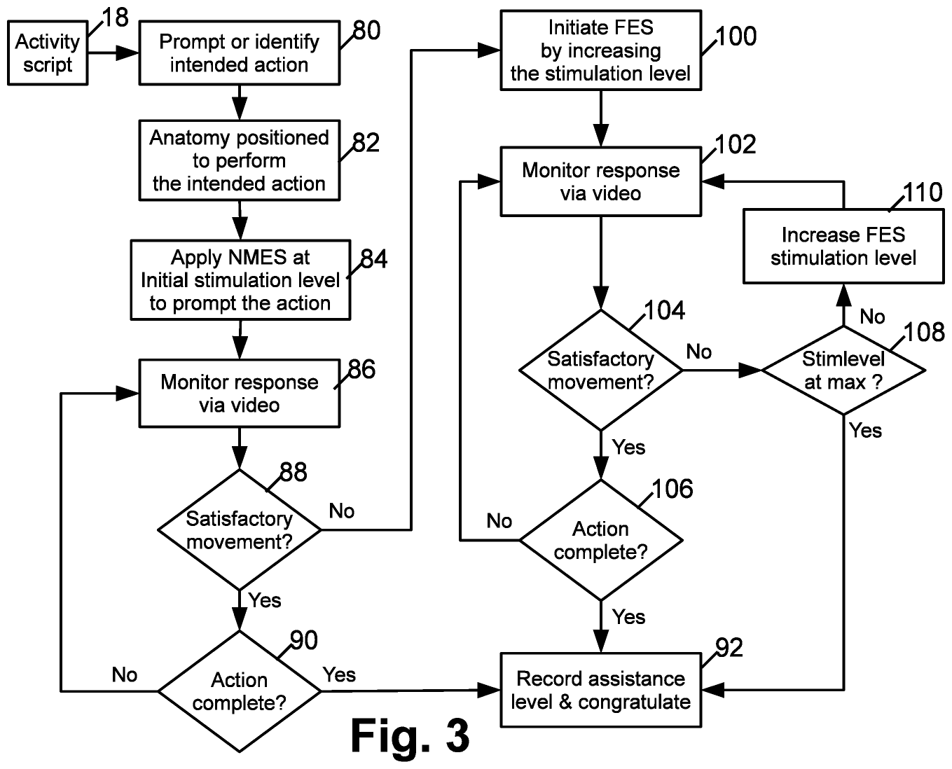

With reference to FIG. 3, a volitional motor control relearning process for relearning volitional motor control to perform an action, including ramping of the NMES, is illustrated in additional detail. In an operation 80, the intended action is identified automatically based on video monitoring of the person (e.g. as described with reference to operation 40 of FIGS. 1 and 2) or the intended action is identified by a prompt issued by the computer 14 in accord with the activity script 18. The prompt may be issued by displaying the prompt on the display 16, and/or by audibly presenting the prompt using a loudspeaker, and/or by another human-perceptible prompt. In an operation 82, the anatomy is positioned to perform the action (if it is not already in position to do so). The operation 82 may be performed by the person P undergoing the relearning if he or she can do so. For example, if the person P has a neurological disorder producing a hand disability but has volitional motor control of the arm, then the person can volitionally move the hand into position to perform an intended action comprising grasping an object. As another example, if both the hand and arm are compromised but the person's other hand and arm are functional then the person can use the other arm/hand to position the compromised hand to perform the grasping action. If the person cannot do either of these things, then a physical therapist can position the hand manually. As another option, the NMES device 20 may be used to move the hand into position by FES, if the NMES device 20 has electrodes positioned to stimulate the appropriate arm muscles. In the case of a leg lift where the person P is seated, there may be no need to perform operation 82 as the leg is already in position to perform the leg lift action.

In an operation 84, NMES is applied at an initial stimulation level that is insufficient to produce functional electrical stimulation of the body part. That is, the initial stimulation level is insufficient to stimulate the muscles to contract to perform the intended action. However, the NMES at this initial stimulation level is enough to provide a prompt to the person P to perform the intended action. For example, the NMES at this initial stimulation level may be sensed by the person P as a tingling sensation or the like in the muscles that should contract to perform the intended action.

In an operation 86, the response to the NMES applied at the initial stimulation level is monitored by the video V (and/or by another sensor or sensors, such as an accelerometer attached to the body part that should move during the intended action or so forth). The purpose of the monitoring operation 86 is to detect whether the person P is able to provide volitional motor control to perform the intended action on his or her own, without FES assistance. Optionally, the operation 86 also monitors the person's volitional motor control by measuring EMG using the EMG amplifier 23. For example, the root mean square (RMS) EMG signal averaged over the electrodes of the NMES device 20 can provide such a volitional motor control metric. To measure the EMG, the NMES applied in operation 84 can be pulsed, with EMG measurements being performed during intervals between the NMES pulses. In some contemplated embodiments, the monitoring operation 84 utilizes EMG in conjunction with hand positional sensors such as the video V, IMU sensor(s) secured to the hand and/or object, bend sensors secured to fingers of the hand H, or so forth, to identify how much effort the user is providing. In a decision 88 it is determined based on the monitoring 86 whether satisfactory volitionally controlled movement is occurring. If so, then flow passes to a decision 90 where it is determined whether the intended action is complete. If not, then flow passes back to the monitoring 86 to continue the monitoring 86 thereby providing monitoring in real-time as the person P attempts to perform the intended action under volitional motor control alone, without FES support. If the person successfully completes the intended action under volitional motor control alone this will be detected at an iteration of the decision 90, at which point flow passes to an operation 92 where the assistance level is recorded. In the instant case in which the person successfully completes the intended action under volitional motor control alone, the assistance level corresponds to the initial stimulation level applied in the operation 84, which was insufficient to produce functional electrical stimulation of the body part. Hence, the recordation operation 92 may record this low initial stimulation level, or may record information such as “No FES applied” or the like. Preferably, the operation 92 also includes congratulating the person P on performing the intended action.

On the other hand, if at the operation 88 satisfactory movement is not detected, flow then passes to provide FES support. This may happen if the person P is unable to produce any volitional motor control to perform the intended action at all, or this may happen if the person P is able to produce some volitional motor control to partially perform the intended action, but the volitionally controlled movement is too slow (e.g., the movement of the body part is slower than some minimal threshold for satisfactory movement) and/or the volitional control produces some movement but that movement either stops before completing the action or is an incorrect movement (e.g., finger uncurling when it needs to curl to perform an intended grasping action).

If the operation 88 determines that satisfactory movement is not being achieved under volitional control alone, then flow passes to an operation 100, where FES is initiated by increasing the NMES stimulation level to a value at which functional electrical stimulation is achieved. The initial stimulation level is preferably sufficient to provide some electrically induced muscle contraction effective for initiating the intended action, but not so much FES as to perform the intended action by FES alone. The goal is for the initially applied FES in combination with volitional muscle control performed by the person P to produce the intended action. In an operation 102 analogous to the operation 86, the response to the initial FES is observed via the video V and/or other sensor feedback. At a decision 104 analogous to the decision 88, it is determined whether satisfactory movement is occurring. If so, then at a decision 106 analogous to the decision 90 it is determined whether the action is complete. If not, then flow passes back to operation 102 to continue monitoring the movement. On the other hand, if at a pass of the decision 106 it is determined that the action is complete, then flow passes to the operation 92 to record the assistance, which here is the stimulation level of the FES applied at operation 100. Preferably, the operation 92 congratulates the person P on achieving the intended action.

On the other hand, if at the operation 104 satisfactory movement is not observed, then flow passes to a decision 108 which determines whether the FES stimulation level can be safely increased, and if so, then at an operation 110 the FES stimulation level is increased and flow passes back to monitoring operation 102 to observe the impact of this increased stimulation level. It will be appreciated that the loop 102, 104, 108, 110 can be performed to implement a ramp of the stimulation level including multiple steps of stimulation level increase increments, until either the intended action is completed as detected at the operation 106 or the maximum safe stimulation level is reached as determined at decision 108. This processing loop enables the FES to be applied only up to a stimulation level that is sufficient to combine with any volitional motor control provided by the person P to perform the intended action.

Notably, the intended action will always be completed, unless the maximum safe stimulation is reached without completing the intended action. If the calibration of the NMES device 20 is accurate, then this latter failure state should never occur unless the volitional motor control is actively opposing the FES to an extent that an unsafe FES stimulation level would be needed to overcome the countering volitional motor control. (An example of this situation would be if the volitional motor control is driving a finger to uncurl when the FES is driving that finger to curl). If the intended action is achieved (with or without FES assistance), then preferably, the operation 92 congratulates the person P on achieving the intended action. In various embodiments, this congratulation may inform the user of the amount of FES assistance; or alternatively the congratulation may omit this information so that the person is encouraged without being informed that the action was partially assisted by FES. In another variant, if the amount of FES assistance provided is less than was required in a previous session performed by the person P then the congratulations may encouragingly note that the person's volitional motor control is improving.

On the other hand, if the FES is ramped up to its maximum safe stimulation level without achieving the intended action as detected at decision 108, then flow again passes to operation 92 which preferably records this failure. The operation 92 in this case may still congratulate the person P on his or her effort, and/or provide other encouragement to the person.

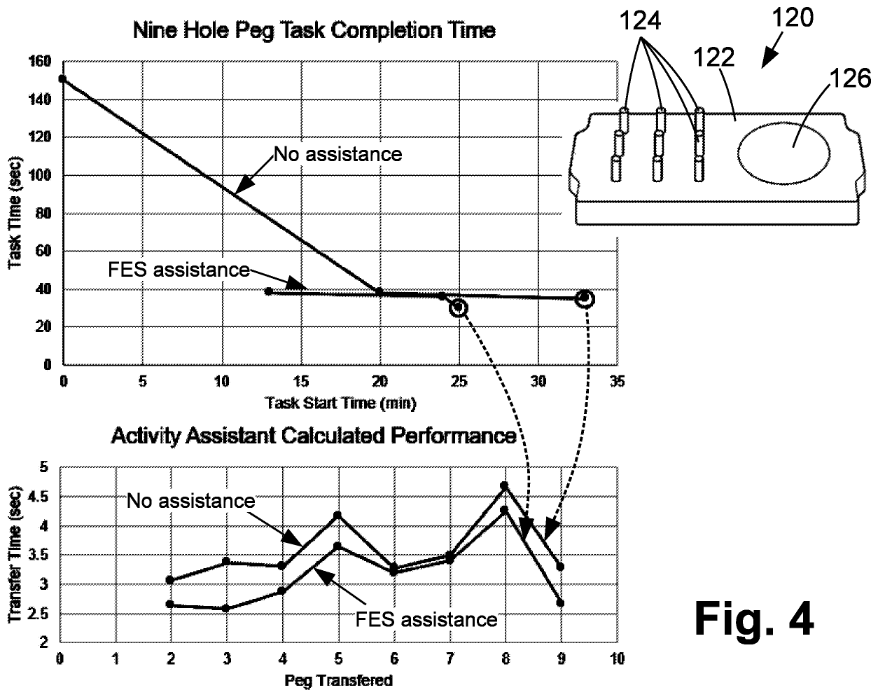

With reference to FIG. 4, an embodiment of the disclosed FES assistance was reduced to practice. The task to perform in this experiment was performed using an apparatus 120 comprising a board 122 with nine openings into which a corresponding nine pegs 124 were placed, and a target area 126. The task entailed the person picking up each peg and 124 moving it to the target area 126. The participant (i.e. person) in this experiment was recovering from a stroke. FIG. 4 further presents plots of the experimental results including a completion time versus task start time plot (top plot) and a plot of the transfer time for each peg (excluding the first peg). The “No assistance” data present the participant's performance with no FES assistance, while the “FES assistance” data present the participant's performance with FES assistance. The FES assistance in this experiment was as follows. Once the computer vision system detected a peg 124 had been picked up and move over or onto the target area 126, the FES sleeve 20 was energized to provide FES to cause the hand to release the peg 124. Such a release action is often challenging for individuals recovering from a stroke. Peg transfer times were calculated by the computer vision and used as indications of performance. FIG. 4 presents experimental task performance over a 35 minute session, with alternation between “No assistance” and “FES assistance”. The “FES assistance” was an “all or nothing” assistance mode. In this mode, FES was not applied at all while the participant picked up a peg 124 and moved it over the target area 126; however, once the peg 124 was over the target area 126 as detected by the computer vision, FES assistance was provided via the FES sleeve 20 with a stimulation level sufficient to cause the hand to release the peg without any volitional release muscular stimulation needed from the participant. Interestingly, despite having only tried this once in the presented experimental results, there was a clear improvement in performance after using FES. The bottom graph of FIG. 4 in particular compares peg transfer times between the final two attempts circled in the top graph. While this experimental test employed “all-or-nothing” FES grip release assistance, it is contemplated to employ a ramping of the FES a disclosed herein to gradually develop the person's ability to release without FES assistance.

In some further contemplated variant embodiments, vagus nerve stimulation (VNS) is employed to further assist in the neuromuscular reeducation. VNS can be applied, for example, using a transcutaneous auricular vagus nerve stimulation (taVNS) device 60 that fits over an ear of the person and is powered by a built-in VNS stimulator circuit (e.g. battery-powered) or has a wired connection to an external VNS stimulator. The taVNS device 60 advantageously provide non-invasive vagus nerve stimulation, and operates on the principle that a branch of the vagus nerve lies close to the surface of the skin in the ear region. In another embodiment, the VNS can be implemented using an implanted VNS stimulator (not shown) having lead wires electrically coupled with the vagus nerve in the neck. For example, the distal ends of the lead wires may be wrapped around the vagus nerve at the carotid sheath. An implanted VNS stimulator advantageously provides strong coupling with the vagus nerve, but requires implantation. VNS can provide positive reinforcement to assist in the neuromuscular reeducation by way of noncognitive electrochemical mechanisms such as release of neuromodulators in response to VNS that reinforces physiological activities occurring concurrently with the VNS. Hence, in some embodiments the operation 84 includes applying VNS using the VNS device 60 concurrently with the NMES to positively reinforce reeducation of the movement being urged by the NMES.

As previously noted, an electronic processor is suitably programmed by instructions stored on a non-transitory storage medium (components not shown) to perform the various processing, data retrieval, and NMES control operations described herein (e.g., operations 30, 32, 34, 36, 40, 44, 46, 52, 54, 56, 62, 64, 70, 72, 80, 82, 84, 86, 88, 90, 92, 100, 102, 104, 106, 108, 110). Additionally, the pre-calibrated stimulation patterns may be stored in a non-transitory storage medium for retrieval in the operation 36. The non-transitory storage medium storing the stimulation patterns may be the same as, or different from, the non-transitory storage medium that stores the instructions executed by the electronic processor. The non-transitory storage medium or media may comprise, by way of nonlimiting illustration: a hard disk drive or other magnetic storage medium or media; a flash memory, CMOS memory, or other electronic storage medium or media; an optical disk or other optical storage medium or media; various combinations thereof, or so forth. The electronic processor may be the electronic processor of the computer 14 or an electronic processor of a server computer or the electronic processors or a cloud-based computing resource, various combinations thereof, and/or so forth. Moreover, while FIG. 1 illustrates the stimulation amplifier 22 as separate from the NMES device 20, the amplifier may alternatively be integrated into the NMES device.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A rehabilitation system comprising:
at least one sensor configured to monitor movement of a body part;
a neuromuscular electrical stimulation (NMES) device configured to be worn on the body part and having electrodes arranged to apply NMES to the body part when the NMES device is worn on the body part; and
an electronic processor programmed to:
obtain a stimulation pattern that when applied to the body part by the NMES device is effective to cause the body part to perform an intended action;
apply the stimulation pattern to the body part using the NMES device; and
stop the application of the stimulation pattern to the body part in response to the at least one sensor indicating the body part has performed the intended action.

2. The rehabilitation system of claim 1 wherein the electronic processor is programmed to apply the stimulation pattern to the body part using the NMES device with a ramping stimulation level by:
applying the stimulation pattern to the body part using the NMES device at an initial stimulation level that is too low to produce functional electrical stimulation of the body part; and
in response to the at least one sensor indicating the body part is not performing the intended action with the stimulation pattern applied to the body part at the initial stimulation level, increasing the stimulation level above the initial stimulation level.

3. The rehabilitation system of claim 2 wherein the increasing of the stimulation level above the initial stimulation level comprising ramping the stimulation level as a function of time.

4. The rehabilitation system of claim 3 wherein:
the stimulation pattern is defined as one or more subsets of electrodes of the NMES device and an electrode group stimulation level for each respective subset of electrodes, and
the electronic processor is programmed to apply the stimulation pattern to the body part using the NMES device with the ramping stimulation level implemented as a ramping of a scaling factor applied to the electrode group stimulation levels.

5. The rehabilitation system of claim 1 wherein the at least one sensor comprises:
a video camera arranged to acquire video of a body part.

6. The rehabilitation system of claim 5 wherein the at least one sensor further comprises the electronic processor further programmed to process the video of the body part to determine whether the body part has performed the intended action.

7. The rehabilitation system of claim 5 wherein the electronic processor is further programmed to:
detect a trigger to perform the intended action based on the video of the body part;
wherein the electronic processor is programmed to apply the stimulation pattern to the body part using the NMES device in response to the detection of the trigger.

8. The rehabilitation system of claim 5 wherein the electronic processor is further programmed to:
identify the intended action based on at least one of (i) the video of the body part and/or (ii) EMG acquired from the body part.

9. The rehabilitation system of claim 1 wherein the electronic processor is further programmed to:
present a prompt of an activity script indicating the intended action.

10. The rehabilitation system of claim 1 wherein the electronic processor is further programmed to record at least a maximum stimulation level applied to the body part using the NMES device.

11. The rehabilitation system of claim 1 wherein the electronic processor is programmed to obtain the stimulation pattern from a non-transitory storage medium using a look-up table associating intended actions with corresponding stimulation patterns.

12. The rehabilitation system of claim 1 wherein the body part is a hand and the NMES device comprises an NMES sleeve configured to be worn on an arm and/or hand.

13. A rehabilitation method comprising:
obtaining a stimulation pattern that when applied to a body part by a neuromuscular electrical stimulation (NMES) device is effective to cause the body part to perform an intended action;
applying the stimulation pattern to the body part using the NMES device, wherein the applying includes increasing a stimulation level at which the stimulation pattern is applied to the body part with increasing time;
during the applying, acquiring video of the body part;
monitoring the body part during the applying by analysis of the video of the body part performed by an electronic processor; and
automatically stopping the applying in response to the monitoring indicating the body part has performed the intended action.

14. The rehabilitation method of claim 13 wherein:
the stimulation pattern comprises one or more subsets of electrodes of the NMES device and an electrode group stimulation level for each respective subset of electrodes; and
the increasing of the stimulation level with increasing time comprises increasing a scaling factor applied to the electrode group stimulation levels over time.

15. The rehabilitation method of claim 13 further comprising:
detecting a trigger to perform the intended action by analysis of the video of the body part performed by the electronic processor;
wherein applying of the stimulation pattern to the body part using the NMES device is performed in response to detecting the trigger.

16. The rehabilitation method of claim 13 further comprising:
identifying the intended action by analysis of the video of the body part and/or EMG acquired from the body part performed by the electronic processor.

17. The rehabilitation method of claim 13 further comprising:
presenting a prompt of an activity script indicating the intended action.

18. A non-transitory storage medium storing instructions readable an executable by an electronic processor to perform a rehabilitation method including:
applying a stimulation pattern to a body part using a neuromuscular electrical stimulation (NMES) device;
during the applying, analyzing video of the body part to determine whether the body part has performed an intended action; and
automatically stopping the applying in response to the analysis of the video indicating the body part has performed the intended action.

19. The non-transitory storage medium of claim 18 wherein the applying includes:
applying the stimulation pattern at an initial stimulation level that is insufficient to produce functional electrical stimulation of the body part; and
in response to the analysis of the video during the applying of the stimulation pattern at the initial stimulation level indicating the body part has not performed the intended action, applying the stimulation pattern at a stimulation level that is higher than the initial stimulation level and that is sufficient to produce functional electrical stimulation of the body part.

20. The non-transitory storage medium of claim 19 wherein the applying of the stimulation pattern at a stimulation level that is higher than the initial stimulation level and that is sufficient to produce functional electrical stimulation of the body part includes ramping up the stimulation level to increase the functional electrical stimulation of the body part over time.
